(19) United States
(12) Patent Application Publication
(10) Pub. No.: US 2014/0243748 A1
(43) Pub. Date: Aug. 28, 2014

(54) IV Backpack

(71) Applicant: Talal A. Kayyal, Houston, TX (US)

(72) Inventor: Talal A. Kayyal, Houston, TX (US)

(21) Appl. No.: 14/194,667

(22) Filed: Feb. 28, 2014

Related U.S. Application Data
(60) Provisional application No. 61/771,071, filed on Feb. 28, 2013.

(51) Int. Cl. A61M 5/14; A61M 25/02

(52) U.S. Cl. CPC A61M 5/1414; A61M 25/02
USPC 604/179

(57) ABSTRACT

An IV backpack comprising: a one or more straps, a IV container, and a one or more clasps; said one or more clasps attaching said IV container to said one or more straps with a one or more supports; with said IV Backpack attached to a patient, said IV container is positioned above an IV entry point on said patient; said IV container holds an IV bag; an IV line attaches said IV bag to said patient at said IV entry point; and said one or more straps attach to a shoulders of said patient and hold said IV container up and behind said patient.

### BACKGROUND

This application is a nonprovisional application filed on the basis of earlier filed provisional U.S. Patent Application No. 61/771,071, filed on 2013 Feb. 28.

This disclosure describes an IV Backpack. None of the known prior inventions or patents in this art space are seen to describe the improvements set out by this disclosure and attached claims. Accordingly, an improved IV Backpack is presented.

### SUMMARY OF THE INVENTION

An IV backpack comprising: a one or more straps, a IV container, and a one or more clasps; said one or more clasps attaching said IV container to said one or more straps with a one or more supports; with said IV Backpack attached to a patient, said IV container is positioned above an IV entry point on said patient; said IV container holds an IV bag; an IV line attaches said IV bag to said patient at said IV entry point; and said one or more straps attach to a shoulders of said patient and hold said IV container up and behind said patient.

### BRIEF DESCRIPTION OF THE DRAWINGS

FIGS. 1A and 1B illustrate a perspective overview and an elevated side view of an IV Backpack assembled on a patient.

FIGS. 2A, 2B, 2C, 2D and 2E illustrate a front perspective overview, a back perspective overview, a side perspective overview and a back view of said IV Backpack.

FIGS. 3A and 3B illustrate an isolated perspective overview and a back perspective overview of said IV Backpack.

### DETAILED DESCRIPTION

Described herein is an IV Backpack. The following description is presented to enable any person skilled in the art to make and use the invention as claimed and is provided in the context of the particular examples discussed below, variations of which will be readily apparent to those skilled in the art. In the interest of clarity, not all features of an actual implementation are described in this specification. It will be appreciated that in the development of any such actual implementation (as in any development project), design decisions must be made to achieve the designers' specific goals (e.g., compliance with system- and business-related constraints), and that these goals will vary from one implementation to another. It will also be appreciated that such development effort might be complex and time-consuming, but would nevertheless be a routine undertaking for those of ordinary skill in the field of the appropriate art having the benefit of this disclosure. Accordingly, the claims appended hereto are not intended to be limited by the disclosed embodiments, but are to be accorded their widest scope consistent with the principles and features disclosed herein.

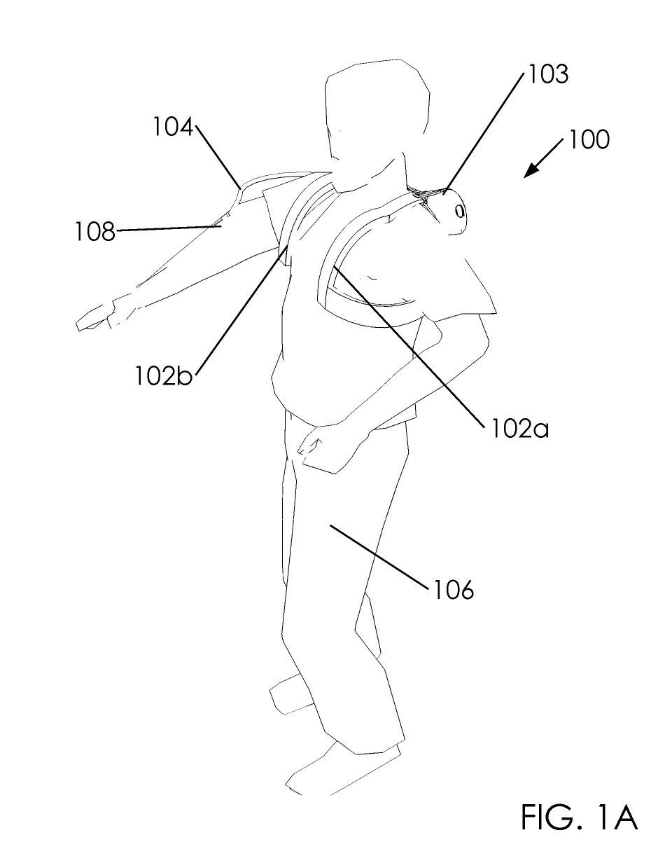

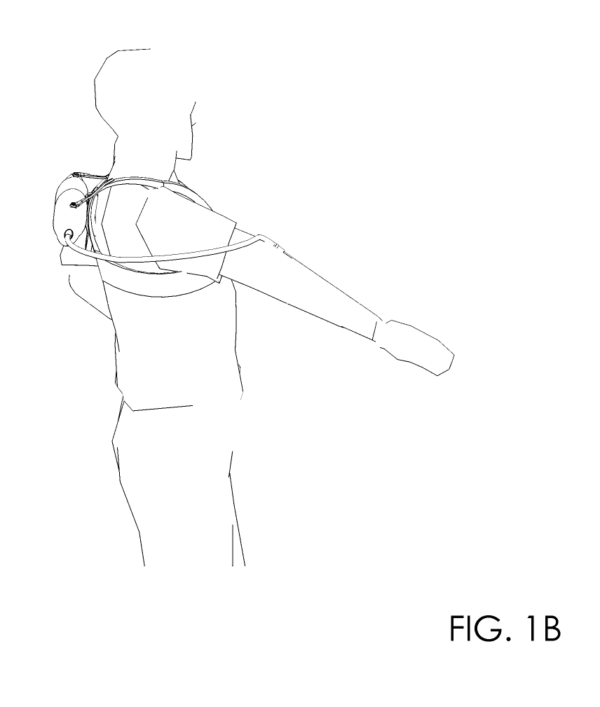

FIGS. 1A and 1B illustrate a perspective overview and an elevated side view of an IV backpack 100 assembled on a patient 106. In one embodiment, said IV backpack 100 can compromise a one or more straps. In one embodiment, said one or more straps can compromise a first strap 102a and a second strap 102b. In one embodiment, said one or more straps can comprise a material made of a plastic, metal or fabric. In one embodiment, said IV backpack 100 can compromise an IV container 103 with an IV line 104 attaching said IV container 103 to an IV entry point 108 on said patient 106. In one embodiment, said IV container 103 can hold an IV bag which can be attached to said IV line 104 and ultimately to said patient 106, as is known in the art.

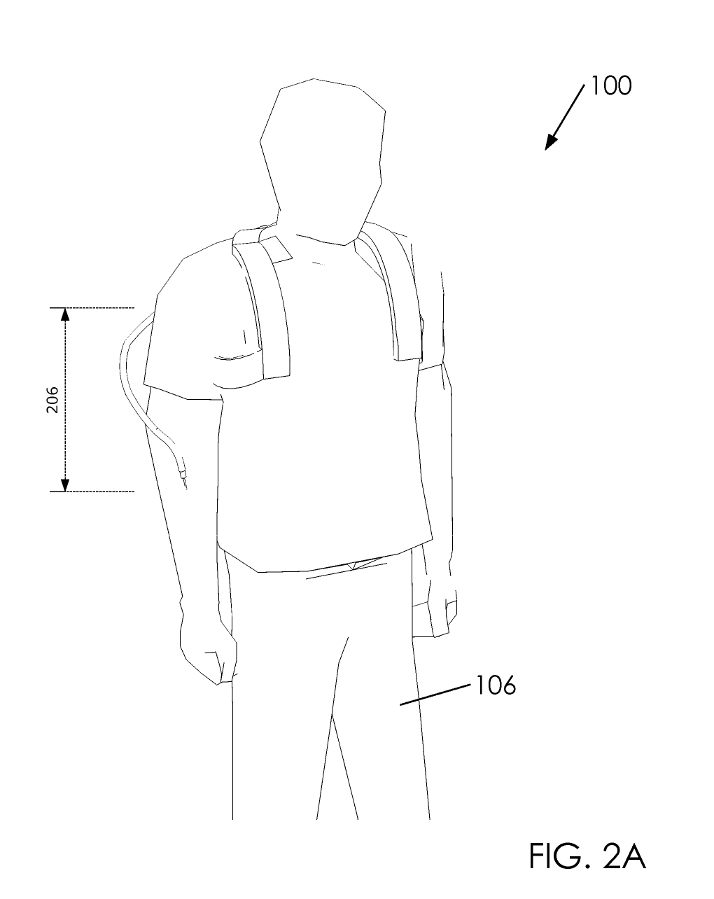

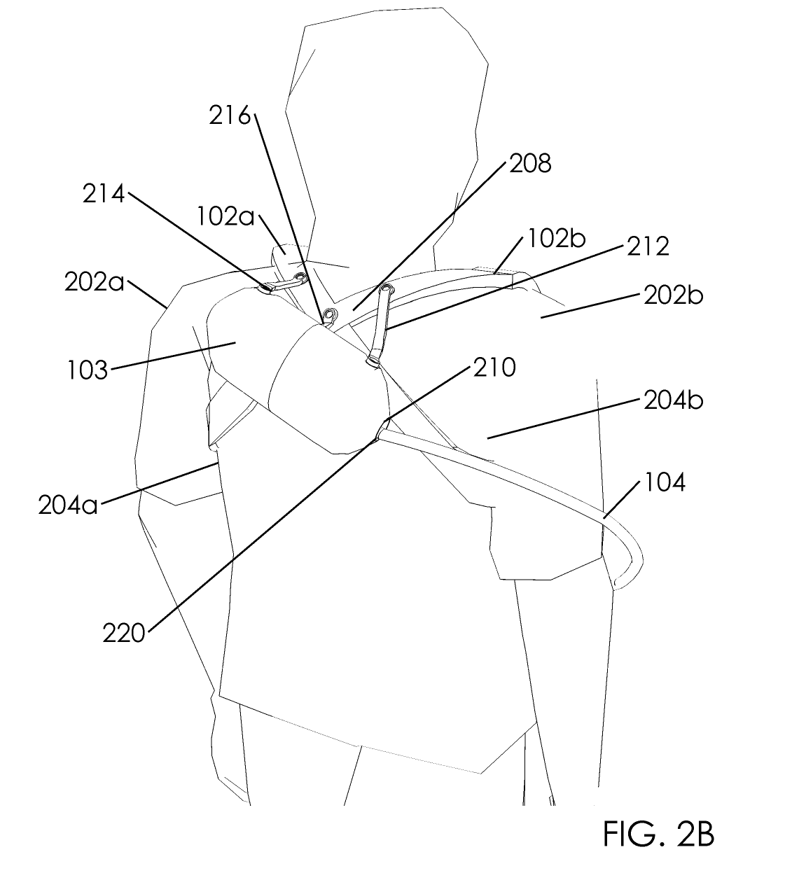

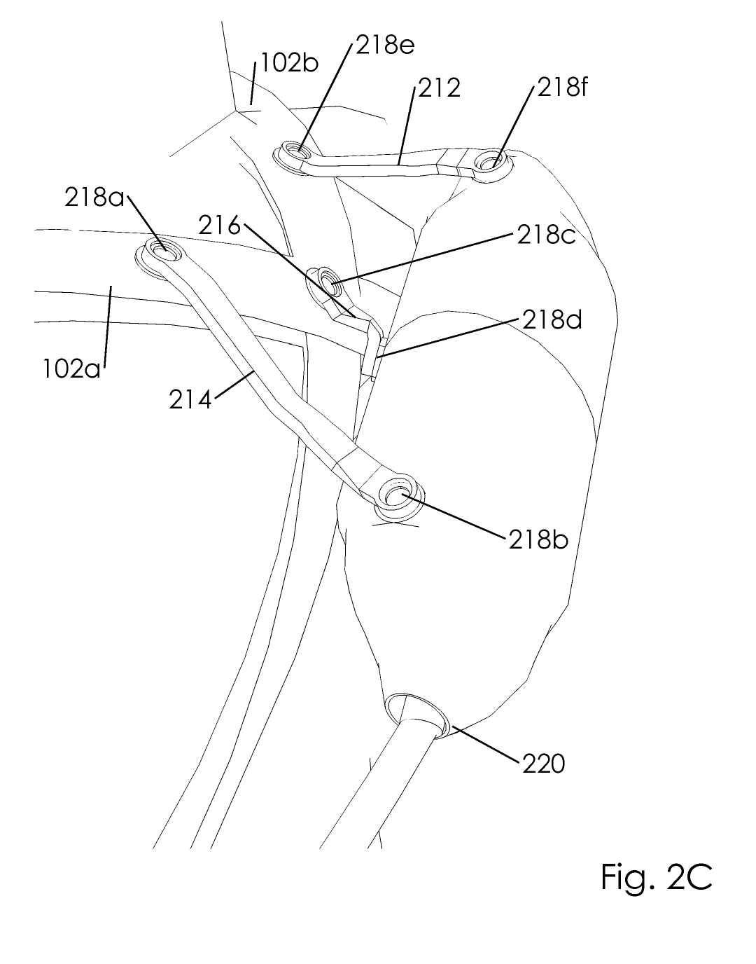

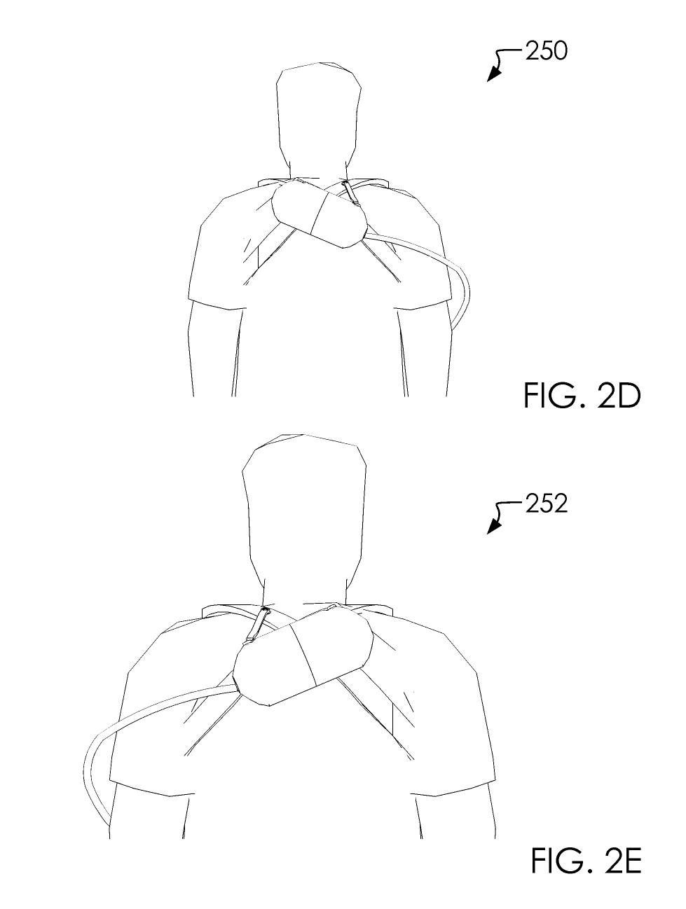

FIGS. 2A, 2B, 2C, 2D and 2E illustrate a front perspective overview, a back perspective overview, a side perspective overview and a back view of said IV backpack 100. In one embodiment, said patient 106 can have their arms hanging downward or lift them upward, as shown in FIG. 2A. In one embodiment, said IV backpack 100 can move with said patient 106 while said patient 106 is recuperating or receiving treatment. In one embodiment, with said IV backpack 100 attached to said patient 106, a draining distance 206, whereby said IV entry point 108 (illustrated in FIG. 2B) can be below an exit point 210 (illustrated in FIG. 2B). Thus, gravity pulls a liquid to said IV entry point 108, through said IV line 104 from said IV bag in said IV container 103 to said patient 106, as is known in the art.

In one embodiment, said patient 106 can comprise a one or more shoulders and a one or more arm pit. In one embodiment, said one or more shoulders can compromise a first should shoulder 202a and a second shoulder 202b. In one embodiment, said one or more arm pit can comprise a first arm pit 204a and a second arm pit 204b.

In one embodiment, said IV backpack 100 can compromise a crossing point 208, whereby said first strap 102a and said second strap 102b intersect. In one embodiment, said IV backpack 100 can compromise a first support 212, a second support 214 and a third support 216. In one embodiment, said IV backpack 100 can compromise a one or more clasps. In one embodiment, said one or more clasps can compromise a first clasp 218a, a second clasp 218b, a third clasp 218c, a fourth clasp 218d, a fifth clasp 218e and a sixth clasp 218f. In one embodiment, said one or more clasp can be used to attach said IV container 103 to said one or more straps. In one embodiment, said IV backpack 100 can compromise a sleeve 220 that can be made of a stiff material, such as plastic, whereby said sleeve 220 can help alleviate an IV bag from cutting off a fluid flow.

In one embodiment, said IV backpack 100 can be reversible wherein said IV backpack 100 can be worn in a configuration A 250 (illustrated at top of FIG. 2D) or a configuration B 252 (illustrated at bottom of FIG. 2D), whichever is suitable for said patient 106. In one embodiment, said IV backpack 100 can have different configurations so that you can have said IV line 104 in a right or left arm depending on circumstances of said patient 106, as would be obvious to one in the art.

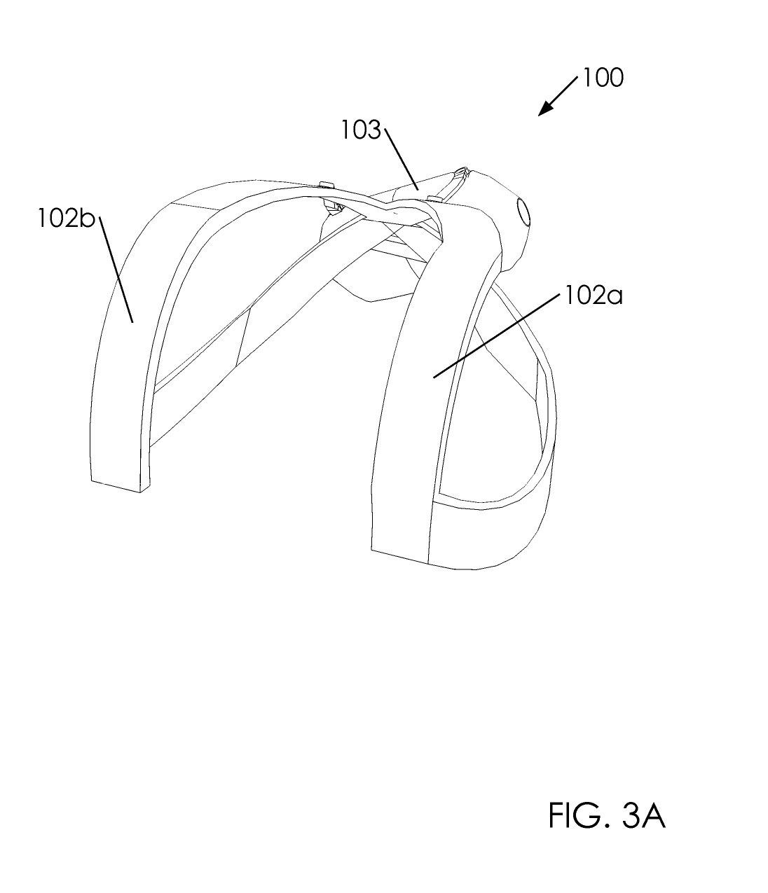

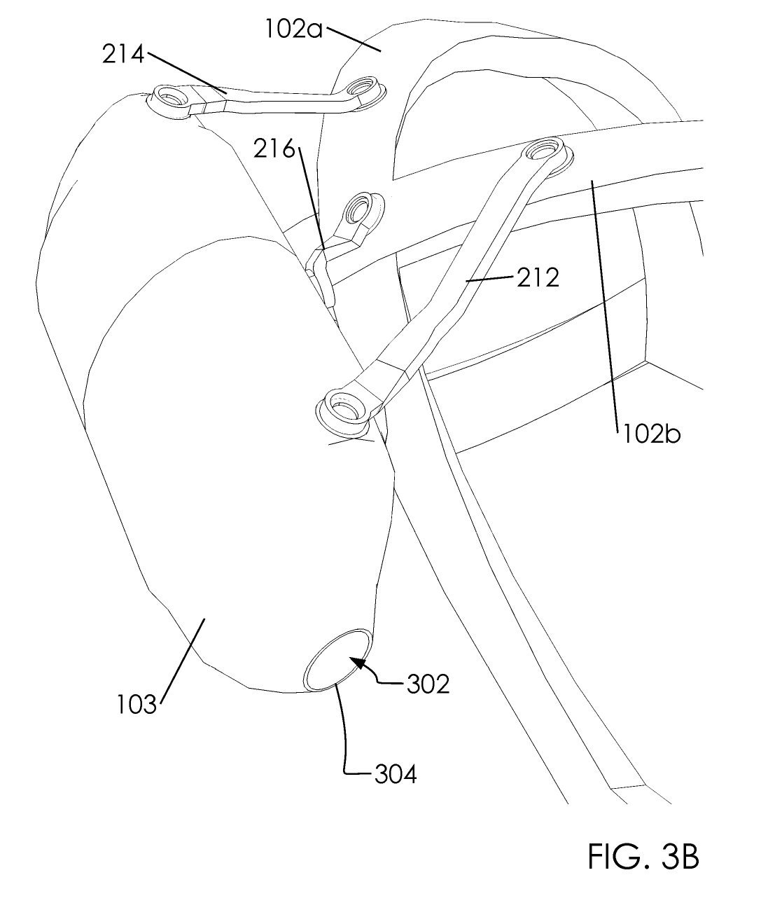

FIGS. 3A and 3B illustrate an isolated perspective overview and an isolated back perspective overview of said IV backpack 100. As illustrated, said IV container 103 can comprise a compartment 302 capable of holding said IV bag (not illustrated, but well-known in the art). In one embodiment, said compartment 302 can open and close at a mouth 304 having an elastic or drawstring opening capable of releaseably holding said IV bag.

Various changes in the details of the illustrated operational methods are possible without departing from the scope of the following claims. Some embodiments may combine the activities described herein as being separate steps. Similarly, one or more of the described steps may be omitted, depending upon the specific operational environment the method is being implemented in. It is to be understood that the above description is intended to be illustrative, and not restrictive. For example, the above-described embodiments may be used in combination with each other. Many other embodiments will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.”

1. An IV backpack comprising:
a one or more straps, a IV container, and a one or more clasps;
said one or more clasps attaching said IV container to said one or more straps with a one or more supports;
with said IV Backpack attached to a patient, said IV container is positioned above an IV entry point on said patient;
said IV container holds an IV bag;
an IV line attaches said IV bag to said patient at said IV entry point; and
said one or more straps attach to a shoulders of said patient and hold said IV container up and behind said patient.

2. The IV backpack of claim 1 wherein:
said one or more straps comprise a first strap and a second strap;
said first strap wraps around a first should shoulder of said patient; and
said second strap wraps around a second should shoulder of said patient.

3. The IV backpack of claim 2 wherein:
said one or more straps cross behind said patient at a crossing point.

4. The IV backpack of claim 1 wherein:
said one or more clasps and said one or more supports suspend said IV container with said IV bag from said one or more straps.

5. The IV backpack of claim 1 wherein:
said IV container comprises a compartment capable of releaseably holding said IV bag.

6. The IV backpack of claim 1 wherein:
said IV container comprises a mouth capable of releaseably holding said IV bag.

7. The IV backpack of claim 6 wherein:
said mouth is elastic.

8. The IV backpack of claim 6 wherein:
said mouth is a drawstring.
